(19) United States
(12) Patent Application Publication
(10) Pub. No.: US 2003/0134411 A1
(43) Pub. Date: Jul. 17, 2003

(54) Cell loading device

(75) Inventors: Jens L. Petersen, Hellerup (DK); Bruno M. Giannetti, Bonn (DE)

Correspondence Address: Louis W. Beardell, Jr., Esquire, Morgan, Lewis & Bockius, L.L.P., 1701 Market Street, Philadelphia, PA, 19103, US

(73) Assignee: Verigen Transplantation Service International (VTSI) AG

(21) Appl. No.: 10/047,571

(22) Filed: Jan. 15, 2002

(51) Int. Cl. C12M 3/00

(52) U.S. Cl. 435/289.1; 435/288.3; 435/307.1

(57) ABSTRACT

A cell loading device including a first member having at least one elevated surface and a second member having an opening configured to accommodate the elevated surface of the first member when the second member is positioned adjacent the first member.

### BACKGROUND

More than 500,000 arthroplastic procedures and total joint replacements are performed each year in the United States. Approximately the same numbers of similar procedures are performed in Europe. Included in these numbers are about 90,000 total knee replacements and around 50,000 procedures to repair defects in the knee per year (In: Praemer A., Furner S., Rice, D. P., Musculoskeletal conditions in the United States, Park Ridge, Ill.: American Academy of Orthopaedic Surgeons, 1992, 125). A method and device for growing cartilage for regeneration-treatment of cartilage would be most useful, because treatment could be performed at an earlier stage of a joint damage, thus reducing the number of patients needing artificial joint replacement surgery. With such preventative methods of treatment, the number of patients developing osteoarthritis would also decrease.

The articular chondrocytes are specialized mesenchymal derived cells found exclusively in cartilage. Cartilage is an avascular tissue whose physical properties depend on the extracellular matrix produced by the chondrocytes. During endochondral ossification, chondrocytes undergo maturation leading to cellular hypertrophy, characterized by the onset of expression of type X collagen (Upholt, W. B. and Olsen, R. R., In: Cartilage Molecular Aspects (Hall, B & Newman, S, Eds.) CRC Boca Raton 1991, 43; Reichenberger, E. et al., Dev. Biol. 1991, 148, 562; Kirsch, T. et al., Differentiation, 1992, 52, 89; Stephens, M. et al., J. Cell Sci. 1993, 103, 1111).

### SUMMARY OF THE INVENTION

The instant invention provides a device that enables one to place a material on a particular portion of a surface of a membrane and prevent the material from contacting other portions of the surface. In particular, the instant invention comprises a device that enables one to place a material, such as cells, on a particular portion of a membrane surface such that the material remains adjacent the membrane surface, and preferably, adheres to the membrane.

In one embodiment, the device includes a first member having at least one elevated or raised platform surface and a second member having an opening configured to accommodate the elevated platform surface of the first member when the second member is positioned adjacent the first member.

In another embodiment, the first member is a flat surface having a raised platform portion. The raised portion of the first member is also configured to optionally have a suitable biodegradable membrane, for example a collagen membrane, readily obtainable from Geistlich Pharma AG, Division Biomaterials, Bahnhofstrasse 40, CH-6110 Wolhusen, Switzerland, placed thereon. A membrane suitable for use in the device of the present invention should not suppress cell growth.

In one embodiment, the second member includes a flat surface having an opening that has a shape and size that corresponds to the shape and size of the raised portion of the first member. Accordingly, when combined together, the elevated platform surface of the first member fits into the opening of the second member.

In yet another embodiment of the first member and the second member, one of the members has an elevated locking surface and the other member has a locking surface opening. When combined together, the elevated locking surface fits into the locking surface opening.

In one embodiment, the instant invention also provides a method of securing a surface for cell loading including the steps of placing into a vessel a first member having at least one elevated platform surface and positioning a membrane on the elevated platform surface of the first member. A second member having an opening configured to accommodate the elevated platform surface of the first member, is placed adjacent the first member. The elevated platform surface and the membrane are inserted into the second member such that the first member and the second member are removably connected and the membrane is secured between the first and second members.

One or more of the embodiments set forth in the present application may be combined with one or more other embodiments to form a device according to the present invention. Further, one or more of the limitations described with relation to one of the first and second members, may be incorporated into, and/or used with, either of the first or second member. For example, the locking mechanism and handling openings (described below) may be incorporated into either of the first or second members.

### BRIEF DESCRIPTION OF THE DRAWING

FIG. 1 is a top view of a first member according to the present invention, having an elevated platform surface;

FIG. 2 is a bottom view of a second member according to the present invention, having an opening that is shaped to accommodate an elevated platform surface of the first member shown in FIG. 1;

FIG. 3 is a cross-section along line A-A of the second member shown in FIG. 2;

FIG. 4 is a side view of the first member and the second member shown in FIG. 1 and FIG. 2, respectively, each according to the present invention, with the opening of the second member accommodating the elevated platform surface of the first member;

FIG. 5 is a top view of an alternative embodiment of a first member according to the present invention, having an elevated platform surface with a plurality of openings positioned on the elevated platform surface;

FIG. 6 is a bottom view of an alternative embodiment of a second member according to the present invention, having an opening that is shaped to accommodate the elevated platform surface of the first member shown in FIG. 5;

FIG. 7 shows a side view of the first member depicted in FIG. 1 in a container;

FIG. 8 is a cross-section along line B-B of the second member shown in FIG. 6.

### DETAILED DESCRIPTION OF THE INVENTION

The present invention is directed to a device including a first member and a second member. When combined, the separate members of the present invention form a device for storing, growing, handling and/or transporting a membrane carrying cells positioned on the membrane. In one embodiment, the membrane and cells are for use as an implantable article. In another embodiment, the device according to the present invention may be used in combination with a container, such as the container disclosed in U.S. patent application Ser. No. 09/852,241, filed May 9, 2001, the entire content of which is hereby incorporated by reference. The container contains a cellular growth medium, such as that set forth in U.S. Pat. No. 5,759,190, the entire content of which is hereby incorporated by reference.

Accordingly, if the device is used with a membrane and placed in a storage vessel with cells positioned adjacent a surface of the membrane (also referred to in some embodiments as “an edge of the membrane”), the instant invention is useful to facilitate the growth and storage of cells, and in particular chondrocytes. The present invention is also useful to 1) maintain the size of a membrane after cell loading by limiting expansion and contraction of a membrane upon contact of the membrane with a cell growth media (also referred to herein as “media”) by providing a boundary for the membrane which is defined by the device and by adjusting the amount of space between the membrane and the device of the present invention, and 2) maintain a membrane in a cell growth media during intra-laboratory moving of the membrane. In one embodiment, the device of the present invention stabilizes the membrane after cell loading. Once stabilized, the membrane can be moved easily about a laboratory, e.g. between a bench, incubator and microscope and back.

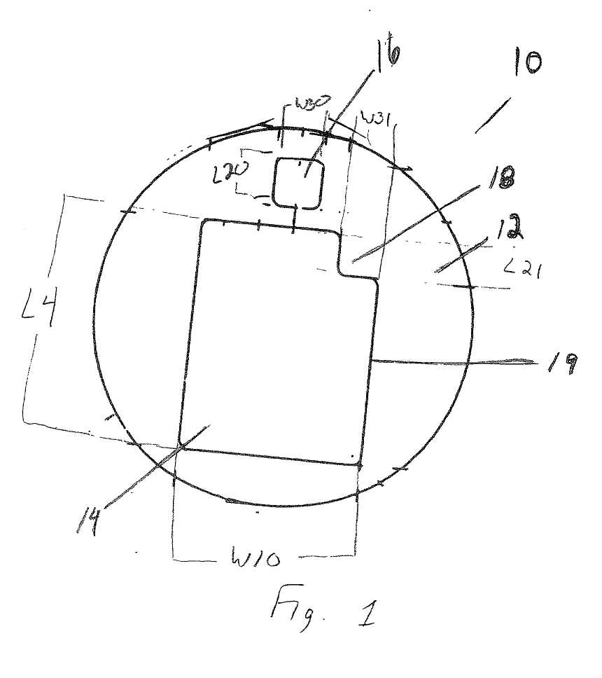

FIG. 1 shows one embodiment of the device according to the present invention. In FIG. 1, first member 10 includes a top surface 12 having at least one elevated platform surface 14, and an optional elevated locking surface 16. Elevated platform surface 14 also optionally includes notch 18, described below.

First member 10 is of any regular or irregular shape and of any size. For example, first member 10 is circular (as shown in FIG. 1), square, rectangular, triangular, or kidney shaped. The overall shape of first member 10 varies depending on the particular use of the device according to the present invention and/or the use, size and/or shape of a membrane used in conjunction with the device, as indicated above and described in more detail below. In one embodiment, the shape and size of a container or vessel into which the device according the present invention is placed dictates the size and shape of first member 10.

In one embodiment, elevated platform surface 14 is a raised portion of first member 10 and has a flat uppermost surface. As shown in FIG. 1, elevated platform surface 14 has a shape that is generally rectangular. However, in other embodiments, elevated platform surface 14 of the present invention is any regular or irregular shape depending on the use of the device or the use, size and/or shape of a membrane used in conjunction with the device.

Further, elevated platform surface 14 is positioned anywhere on first member 10 as appreciated by one of skill in the art. For example, in the embodiment shown in FIG. 1, elevated platform surface 14 has a rectangular shape which is centrally positioned on first member 10.

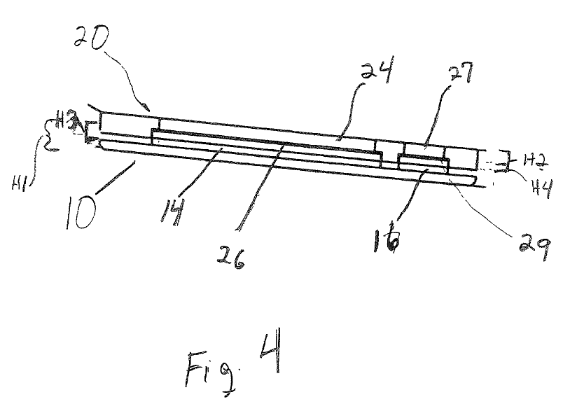

In a particular embodiment, first member 10 has an overall height of 0.25 to 25 mm (described further with respect to FIG. 4), preferably about 3 mm, and if circular it has a radius of about 1 to 6 cm, or more. In other particular embodiments, first member 10 has dimensions that are limited by the dimensions of the vessel into which first member 10 is placed.

Additionally, in particular embodiments, the dimensions of elevated platform surface 14 are typically about 10 to 90 mm long (depicted as L4 in FIG. 1) and 10 to 90 mm wide (depicted as W10 in FIG. 1). Also, in particular embodiments, elevated platform surface 14 extends 0.25 to 25 mm above top surface 12 of first member 10 (also described further with respect to FIG. 4). In general, the dimensions of the vessel or container intended to store the device according to the present invention limit the dimensions of elevated platform surface 14 and first member 10.

In an embodiment where elevated platform surface 14 includes notch 18, notch 18 can be positioned anywhere along edge 19 of elevated platform surface 14. In one embodiment, as shown in FIG. 1, notch 18 is positioned along edge 19 at a corner of a rectangularly shaped elevated platform surface 14. In one embodiment, notch 18 may be placed at a corner of elevated platform surface 14 in close proximity to optional elevated locking surface 16. Notch 18 provides an advantage of facilitating the placement and removal of a membrane positioned on elevated platform surface 14 by allowing a user to pick up a free end of a membrane positioned on elevated platform surface 14 as well as provide other advantages, as described below. In particular embodiments of the present invention, the dimensions of notch 18 (if square or rectangular in shape) are a width W31 of about 5 to 15 mm and a length L21 of about 5 to 15 mm.

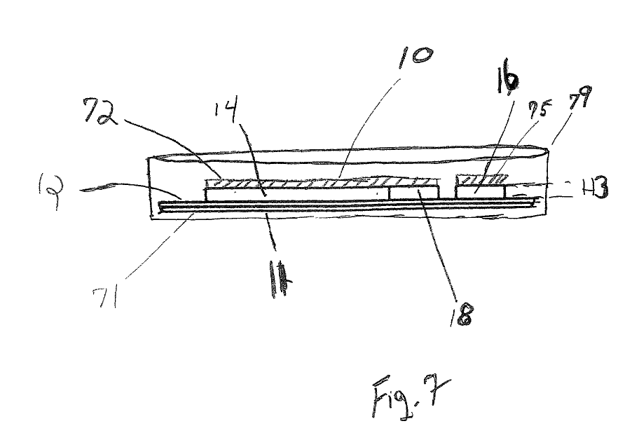

In an embodiment where membrane 72 (shown in FIG. 7) is a bilayer membrane, it is important to provide a physical mark or indicator for determining which side of membrane 72 has cells disposed thereon because the opposing sides of membrane 72 look similar upon visual inspection. To solve this problem, removal of a portion of membrane 72 that is over notch 18 orients membrane 72 in three-dimensional space by physically marking membrane 72. Specifically, in an embodiment wherein platform surface 14 is roughly rectangular in shape or otherwise irregularly shaped, when a portion of membrane 72 is marked in a manner described above, membrane 72 can be easily removed and replaced on platform surface 14 in its original orientation by aligning membrane 72 with notch 18. Specifically, when the corresponding physical mark of membrane 72 aligns with notch 18, membrane 72 is in the proper orientation with a predetermined side of membrane 72 facing the desired direction.

In one embodiment of a device according to the present invention, first member 10 also has an optional elevated locking surface 16 (also referred to herein as locking surface 16). Similar to elevated platform surface 14, elevated locking surface 16 is of any regular or irregular shape. However, in some embodiments the shape and dimensions of elevated locking surface 16 corresponds to the shape and dimensions of notch 18, such that elevated locking surface 16 has approximately a length L20 (shown in FIG. 1) and a width W30 (shown in FIG. 1). In one embodiment, elevated locking surface 16 is shaped such that it locks and connects second member 20 to first member 10 such that second member 20 cannot rotate relative to first member 10, thereby preventing accidental damage to a membrane and/or cells positioned on a membrane that is on elevated platform surface 14. In another embodiment, elevated locking surface 16 is shaped such that it does not prevent rotation of second member 20 about first member 10.

In another embodiment, locking surface 16 can support a portion of membrane 72. Specifically, locking surface 16 can support the portion of membrane 72 that is initially over notch 18 which is then removed to mark membrane 72 (as described above) wherein the removed portion forms membrane 75. Membrane 75 can then be placed on elevated locking surface 16, as also shown in FIG. 7.

As described above, in some embodiments the dimensions of elevated locking surface 16 approximate the dimensions of notch 18 such that the dimensions of membrane 75 then approximate the dimensions of elevated locking surface 16. Locking surface 16 and notch 18 can be any regular or irregular shape. For example, locking surface 16 and notch 18 are round, square, rectangular, triangular, etc.

Placing membrane 75 on elevated locking surface 16 has the advantage of providing a control membrane separate from membrane 72. Specifically, because membrane 75 on elevated locking surface 18 is typically exposed to the same conditions as membrane 72 on elevated platform surface 14, if cells are adhered to both membrane 72 and 75, and both membranes have a similar cell density, membrane 75 and cells thereon can be analyzed to determine the condition of membrane 72 and cells on membrane 72 without disturbing membrane 72 or cells on membrane 72. This information can then be used to generate data regarding the state of the cells on membrane 72 before transport of membrane 72 to a physician and/or implantation of membrane 72 and cells thereon into a patient. Membrane 75, with cells thereon, can also be stored in a separate climate controlled facility to preserve membrane 75 and the cells thereon, which can then be used later in time to determine the physical and chemical state of membrane 72 with cells thereon. In such an embodiment, membrane 75 with cells thereon can function as a quality control indicator for membrane 72 with cells thereon.

In an embodiment of the present invention having optional elevated locking surface 16, elevated locking surface 16 is positioned about 5 to 20 mm away from edge 19 of elevated platform surface 14 and about 5 to 20 mm from the periphery of first member 10 nearest elevated locking surface 16. The dimensions of elevated locking surface 16 are typically about 5 to 15 mm by 5 to 15 mm. Accordingly, in some embodiments, width W31 equals width W30 and length L21 equals length L20.

The height of elevated locking surface 16 extends to or above the uppermost surface of elevated platform surface 14 (described in more detail with respect to FIG. 4). In a preferred embodiment of the present invention, elevated locking surface 16 has a height equal to the height of elevated platform surface 14.

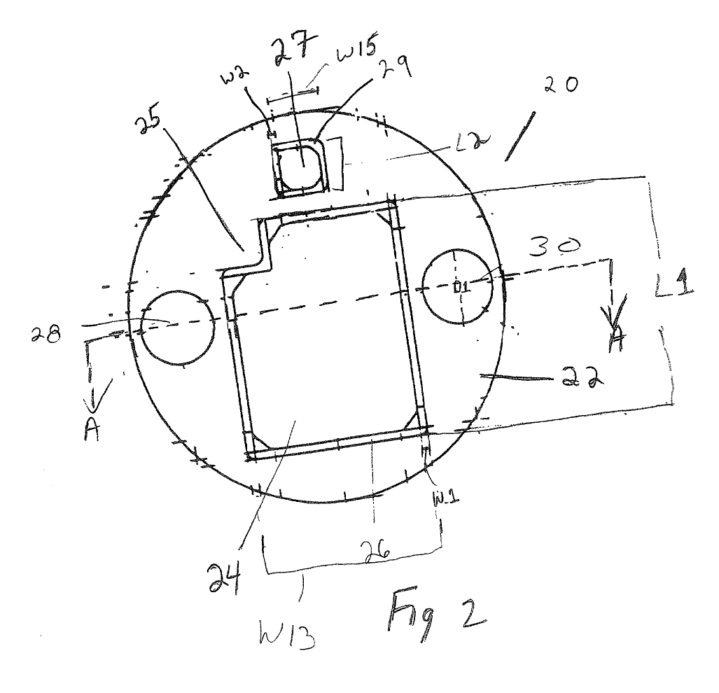

FIG. 2 shows one embodiment of second member 20 according to the present invention. As shown in FIG. 2, second member 20 includes bottom surface 22, platform opening 24, optional locking surface opening 27, and optionally one or more handling openings 28 and 30. Platform opening 24 and locking surface opening 27 also include optional seating surfaces 26 and 29, respectively.

In one embodiment, the overall shape of second member 20 corresponds to the shape of first member 10. For example, in the embodiment shown in FIG. 2, second member 20 is circular to correspond to the circular shape of first member 10, as shown in FIG. 1. In other embodiments, second member 20 is square, rectangular, triangular, kidney-shaped or any other regular or irregular shape depending on the particular use and/or size and/or shape of membrane 72 to be held in the device according to the present invention. In other embodiments, the shape and dimensions of second member 20 may not correspond to those of first member 10.

In practice, as described below, second member 20 of the present invention is positioned adjacent first member 10, such that bottom surface 22 of second member 20 is in close proximity or touching top surface 12 of first member 10. Platform opening 24 of second member 20 is shaped to accommodate elevated platform surface 14 of first member 10. Accordingly, platform opening 24 is round, rectangular, square, kidney-shaped or any other regular or irregular shape, and typically corresponds to the shape of elevated platform surface 14 of first member 10.

In one embodiment which includes notch 18 on first member 10, a corresponding protrusion 25 exists on second member 20. Protrusion 25 is configured to occupy notch 18. In another embodiment, protrusion 25 is shaped to provide the advantage of preventing rotation of second member 20 about first member 10 as well as cover a portion of membrane 72 that is not removed to form membrane 75. In some embodiments, protrusion 25 does not prevent rotation of second member 20 about first member 10. In other embodiments, protrusion 25 does not cover a portion of membrane 72.

In one embodiment, second member 20 has one or more openings in addition to platform opening 24. For example, second member 20 has one or both of a locking surface opening 27 and one or more handling openings 28 or 30 (see FIG. 2). Locking surface opening 27 is an opening configured to accommodate elevated locking surface 16 of first member 10. In one embodiment, locking surface opening 27 has dimensions that approximate the dimensions of elevated locking surface 16.

Handling openings 28 and 30 are openings positioned on second member 20 that facilitate manipulation and placement of second member 20 adjacent or against first member 10. Handling openings 28 and 30 provide certain advantages, including providing increased ease of handling and placement of second member 20 and the overall device and reducing the likelihood of contamination of 1) media in a vessel holding the device according to the present invention, and 2) any membrane or cells that may be placed in the device. In one embodiment, handling openings 28 and 30 are configured to facilitate the use of tongs, calipers or other devices for the manipulation of the second member 20 relative to first member 10, or manipulation of the entire device. Handling openings 28 and 30 have any regular or irregular shape, but preferably are circular with diameters D1 of about 2 to 10 mm. As shown in the embodiment in FIG. 2, handling openings 28 and 30 are circular and positioned on either side of the platform opening 24.

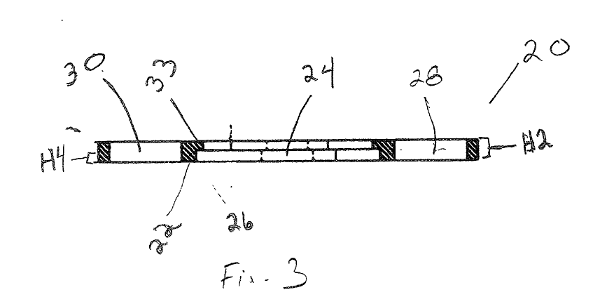

In one embodiment, to ensure that elevated platform surface 14 does not extend beyond the height of second member 20 (described in more detail below with respect to FIG. 4), second member 20 is formed with an optional seating surface 26 around the inside periphery of platform opening 24. With reference to FIGS. 2 and 3, seating surface 26 extends circumferentially around the inside periphery of platform opening 24 between bottom surface 22 and top surface 33 of second member 20. In particular, seating surface 26 provides an area of contact for second member 20 to rest on elevated platform surface 14 of first member 10. Similarly, locking surface opening 27 can also have an optional seating surface 29. Seating surface 29 in locking surface opening 27 performs substantially the same function as seating surface 26 in platform opening 24. This particular arrangement of second member 20 resting on first member 10 is shown generally in FIG. 4, described below.

Additionally, seating surfaces 26 and 29 are positioned anywhere along the inside edge of platform opening 24 and locking surface opening 27, respectively. In particular embodiments, seating surfaces 26 and 29 have widths W1 and W2 of 0.05 to 10 mm. Additionally, widths W1 and W2 can vary at any point along the periphery of the opening. Preferably, seating surfaces 26 and 29 have widths W1 and W2 of 0.5 to 2 mm. Seating surfaces 26 and 29 can have any lengths L1 and L2, respectively, and may be positioned along one or more portions of the periphery of platform opening 24 and locking surface opening 27, respectively.

In some embodiments, length L1 equals or exceeds length L4 (shown in FIG. 1) and length L2 equals or exceeds length L20 (shown in FIG. 1). Similarly, in some embodiments width W15 equals or exceeds width W30 and width W13 equals or exceeds width W10. In an embodiment where one or more of the dimensions L1, L2, W13 and W15 exceed the dimensions of one or more of L4, L20, W10 and W30, respectively, such an arrangement provides the advantage of increasing space between first member 10 and second member 20, thereby permitting membrane 72 to expand easily into that space upon contact with media, as described below. In such embodiments, L1, L2, W13 and W15 exceed the dimensions of one or more of L4, L20, W10 and W30 by 0.5 to 2 mm.

FIG. 3 shows a cross section of second member 20 along line A-A, shown in FIG. 2. Bottom surface 22, top surface 33 and heights H2 and H4 are shown. In this embodiment, seating surface 26 is also shown. The size of platform opening 24 on bottom surface 22 is larger relative to the size of opening 24 on top surface 33, thus forming seating surface 26. Handling openings 28 and 30 are also shown in cross section.

FIG. 4 shows a side view of first member 10 inserted into second member 20. As shown in FIG. 4, elevated platform surface 14 is inserted into platform opening 24. Additionally, elevated locking surface 16 is inserted into locking surface opening 27. FIG. 4 also shows second member 20 resting on first member 10 via contact of elevated platform surface 14 with seating surface 26 and elevated locking surface 16 with seating surface 29.

First member 10 has a height Hi, and second member 20 has a height H2. In this embodiment, the height of elevated platform surface 14 above top surface 12 is shown as height H3 and seating surface 26 has a height H4. Height H1, height H2, height H3 and height H4 are chosen based on the particular use and/or physical or chemical attributes of a membrane held in a device according to the present invention. In a preferred embodiment, height H3 is less than height H2 such that elevated platform surface 14 does not extend through the entire height H2 of second member 20. Alternatively, height H4 is less than H3, such that elevated platform surface 14 does not extend through the entire height H2 of second member 20 and second member 20 rests via seating surfaces 26 and 29 on elevated platform surface 14 and elevated locking surface 16.

In one embodiment, second member 20 has a height H2 and height H4 of about 0.25 mm to 50 mm, preferably about 5 mm each, and first member 10 has a height H1 and height H3 of about 0.25 mm to 50 mm.

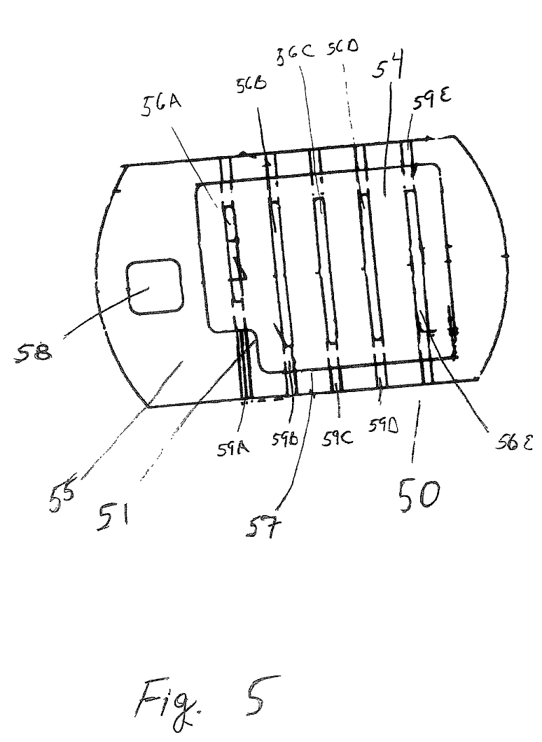

FIG. 5 shows an alternative embodiment of a device according to the present invention. In FIG. 5, first member 50 includes a top surface 55 having at least one elevated platform surface 54, and an optional elevated locking surface 58. Elevated platform surface 54 optionally includes notch 51.

First member 50 is of any regular or irregular shape and of any size. For example, first member 50 is curved rectangular (as shown in FIG. 5), square, circular, triangular, or kidney shaped. The shape of first member 50 varies depending on the particular use of the device according to the present invention and/or the use, size and/or shape of a membrane used in conjunction with the device. As described above with respect to FIG. 1, the shape and size of a container into which the device according the present invention is to be placed also dictates the size and shape of first member 50. However, the curved rectangular shape depicted in FIG. 5 has the advantage of providing additional space in a vessel for additional cell growth media.

In one embodiment, elevated platform surface 54 has at least one opening 56A that extends through the entire thickness first member 50. Elevated platform surface 54 can have at least one opening 56A, and preferably has two or more openings such as openings 56A, 56B, 56C, 56D, 56E, depicted in FIG. 5.

Additionally, in an embodiment shown in FIG. 5, openings 56A-56E may have any shape appreciated by one of skill in the art, and in particular embodiments, the openings are rectangular, square, circular or oblong. In one embodiment, openings 56A-56E are formed by drilling, lasing, cutting, or melting portions of first member 50 or by other methods appreciated by one of skill in the art, such as incorporating the openings into a mold for forming first member 50. The dimensions of openings 56A-56E can vary in different embodiments. In one embodiment, openings 56A-56E are about 0.1 mm to 10 mm in diameter and from about 5 mm to 70 mm in length, depending on the size of platform surface 54.

Openings 56A-56E in first member 50 provide an advantage of forming passages through first member 50. These passages facilitate the loading of cells onto a membrane and/or facilitate the growth or ingrowth of cells onto or into membrane 72. Specifically, as media and cells are contacted with a membrane, such as by pouring the media and cells onto a membrane held in a device of the present invention as described in more detail below, the media passes through membrane 72 and into the passages and through the device. In one embodiment, the rate at which the media passes or diffuses through membrane 72 can vary depending on the density of membrane 72, the density and viscosity of the media and the amount of cells disposed on or in membrane 72.

It is because the media passes through membrane 72 and through the device that the amount of media and cell spillover onto and/or over the device is significantly reduced. Such spillover is undesirable because it can result in cell loss, and in some instances the spillover can result in the adhesion of cells onto unusable or unwanted portions of membrane 72.

Openings 56A-56E also provide an advantage of enhancing cell adhesion to membrane 72. Specifically, after passing through the passages, the media enters the vessel that contains the device of the present invention. However, as the media passes through membrane 72, the cells contained in the media (which are too large in size to pass through membrane 72 with the media) remain adjacent the surface of membrane 72 and are effectively filtered out of the media as the media passes through membrane 72. Additionally, the flow of the media through membrane 72 can also draw a portion of the cells adjacent membrane 72 at least partially into membrane 72, thereby enhancing cell adhesion to membrane 72. In one embodiment, openings 56A-56E increase the amount of nutrients, oxygen, and waste that can diffuse through membrane 72, thereby enhancing ingrowth of cells into membrane 72.

Openings 56A-56E in first member 50 provide another advantage of facilitating nutrient contact with cells already on membrane 72 without disturbing the cells. Specifically, the passages permit media already contained in a vessel holding first member 50 to contact a downward facing side of a membrane placed in a device according to the present invention. Media already contained in a vessel containing the device of the present invention can move upwardly though openings 56A-56E and contact the bottom-facing surface of membrane 72 on platform surface 54. The media then diffuses upwardly through membrane 72 to contact cells positioned on the edge or adjacent membrane 72.

In other embodiments, to facilitate contact between fresh media and a membrane positioned on elevated platform surface 54, channels or grooves can be cut into the bottom surface of first member 50 to connect the passages formed by openings 56A-56E with the media contained in a vessel, thereby permitting communication of the media in a vessel in which the device of the present invention is placed with the bottom side of membrane 72 positioned on elevated platform surface 54 through openings 56A-56E of first member 50. To facilitate contact of media with a membrane positioned on elevated platform surface 54, one or more channels or grooves 59A, 59B, 59C, 59D and 59E, are cut, melted, lased, or molded into first member 50 to more easily permit media in the vessel to contact the bottom side of a membrane positioned on elevated platform surface 54.

In one embodiment, a chamber can be placed under first member 50 and in fluid communication with openings 56A-56E, thereby creating a vacuum on the lower side of first member 50 to enhance the drawing of cells and fresh media into membrane 72 and facilitating additional ingrowth of cells onto and/or into membrane 72. In one such embodiment, the vacuum draws fresh media through openings 59A-59E, thereby facilitating cell growth and/or ingrowth onto and/or into membrane 72.

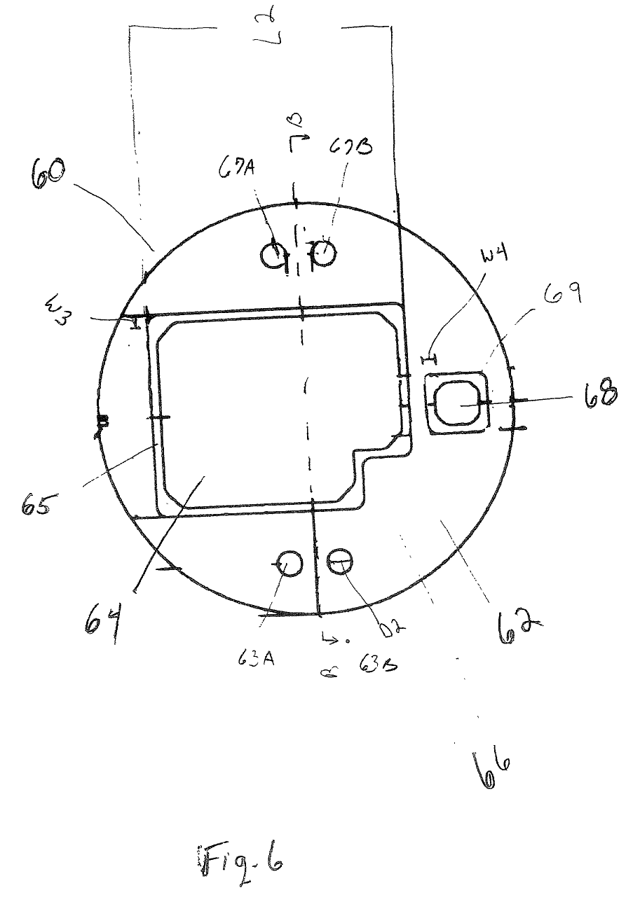

In one embodiment of a device according to the present invention, first member 50 also has a elevated locking surface 58 which is a raised portion that is accommodated by a corresponding locking surface opening 68 of second member 60 (as described below with respect to FIG. 6). Similar to elevated platform surface 54, elevated locking surface 58 is of any regular or irregular shape such that it fits into locking surface opening 68. Additionally, elevated locking surface 58 can have a membrane 75 disposed thereon in the manner described above with respect to FIG. 1. Furthermore, elevated locking surface 58 can also have one or more openings (not shown) to form passages through first member 50 in a manner similar to that described above with respect to platform surface 54.

In one embodiment, elevated platform surface 54 is rectangular, and includes notch 51. Notch 51 is positioned anywhere along edge 57 of elevated platform surface 54, as described with respect to notch 18 shown in FIG. 1 above.

In a particular embodiment, the dimensions of first member 50 are the same as the dimensions set forth above for first member 10, described above. The dimensions and position of elevated locking surface 58 are also similar to those described above with respect to the elevated locking surface 16 depicted in FIG. 1. In particular embodiments of the present invention, the dimensions of notch 51 and elevated locking surface 58 have the same dimensional requirements as notch 18 and elevated locking surface 16, set forth above. In certain embodiments, notch 51 and elevated locking surface 58 (if square or rectangular in shape) are each about 5 to 15 mm by 5 to 15 mm.

The dimensions of elevated platform surface 54, and first member 50, are limited by the dimensions of the vessel or container intended to store the device according to the present invention.

FIG. 6 shows one embodiment of second member 60 according to the present invention. As shown in FIG. 6, second member 60 includes bottom surface 62, platform opening 64, optional locking surface opening 68, and optional handling openings 63A, 63B, 67A and 67B. Platform opening 64 and locking surface opening 68 also include optional seating surfaces 65 and 69, respectively.

In the embodiment shown in FIG. 6, second member 60 is circular. In other embodiments, second member 60 can be any regular or irregular shape and can also be square, rectangular, triangular, kidney-shaped or any other shape, depending on the particular use and/or size and shape of membrane 72 to be held in the device according to the present invention. In other embodiments, the overall shape and dimensions of second member 60 may not correspond identically to that of first member 50.

Similar to the embodiments described above, second member 60 of the present invention is positioned adjacent first member 50, such that bottom surface 62 of second member 60 is in close proximity or touching top surface 55 of first member 50. Platform opening 64 of second member 60 is shaped to accommodate elevated platform surface 54 of first member 50. As described in more detail below, in one embodiment, first member 50 and second member 60 are held together by gravity, thus holding membrane 72 therebetween.

Similar to platform opening 24 described above, platform opening 64 can be any regular or irregular shape, including round, rectangular, square, kidney-shaped or another shape, and typically corresponds to the shape of elevated platform surface 54 of first member 50. In one embodiment which includes notch 51 on first member 50, a corresponding protrusion 66 exists on second member 60 can occupy notch 51.

In one embodiment, second member 60 has openings in addition to platform opening 64. For example, second member 60 has one or both of a locking surface opening 68 and one or more handling opening 63A, 63B, 67A and 67B.

Locking surface opening 68 is an opening configured to accommodate elevated locking surface 58 of first member 50. In one embodiment, locking surface opening 68 has dimensions that approximate the dimensions of elevated locking surface 58.

Similar to the handling openings 28 and 30 described above with reference to FIG. 2, handling 63A, 63B, 67A and 67B are openings positioned on second member 60 that facilitate manipulation and placement of second member 60 adjacent or against first member 50. Handling openings 63A, 63B, 67A and 67B provide certain advantages, including increasing the ease of handling and placement of second member 60 and reducing the likelihood of contamination of 1) media in a vessel holding the device according to the present invention, and 2) any membrane or cells that are placed in the device. In one embodiment, handling openings 63A, 63B, 67A and 67B are configured to facilitate the use of tongs, calipers or other devices for the manipulation of the second member 60. As discussed above, handling openings 63A, 63B, 67A and 67B can have any regular or irregular shape, but preferably are circular with diameters D2 of about 2 to 10 mm. As shown in the embodiment in FIG. 6, handling openings 63A, 63B, 67A and 67B are circular and positioned on either side of the opening 64.

In one embodiment, to ensure that elevated platform surface 54 does not extend through an entire thickness of second member 62, second member 62 is formed with an optional seating surface 65 around the inside periphery of platform opening 64. Locking surface opening 68 can also have an optional seating surface 69. Seating surfaces 65 and 69 can be configured the same as seating surfaces 26 and 29, described above

Additionally, seating surfaces 65 and 69 are positioned at one or more portions anywhere along the inside periphery of platform opening 64 and locking surface opening 68, respectively. In particular embodiments, seating surfaces 65 and 69 have a width W3 and width W4 of 0.05 to 10 mm. Widths W3 and W4 can vary at any point along the periphery of the opening. Seating surfaces 65 and 69 are positioned anywhere along the inside edge of platform opening 64 and locking surface opening 68, respectively.

FIG. 7 shows a side view of the embodiment described above with reference to FIG. 1. In FIG. 7, the embodiment is properly oriented in petri-dish 79, wherein first member 10 has top surface 12, and bottom surface 71 of first member 10, which is in contact with petri-dish 79. Elevated platform surface 14 has a height H3 above top surface 12 and also has notch 18. Elevated locking surface 16 is also clearly shown. Membrane 72, placed on the top of elevated platform surface 14 is also shown and membrane 75, placed on the top of elevated locking surface 16 is also shown.

In one embodiment, membrane 72 has approximately the same length and width dimensions as platform surface 14 and membrane 75 has approximately the same length and width dimensions as elevated locking surface 16. Specifically, the length of membrane 72 approximately equals length L4 and the width of membrane 72 approximately equals width W10 and the length of membrane 75 approximately equals length L20 and the width of membrane 75 approximately equals width W30 (wherein W10, W30, L4 and L20 are shown in FIG. 1). Accordingly, in one embodiment, the dimensions of membrane 72 approximately equal the outer dimensions of the periphery of seating surface 26 and the dimensions of membrane 75 approximately equal the outer dimensions of the periphery of seating surface 29.

Additionally, in some embodiments, the dimensions of membrane 72 and membrane 75 can be smaller than the platform surface 54 and locking surface 58. In some embodiments where membrane 72 and membrane 75 are smaller in dimension than platform surface 54 and locking surface 58, portions of the periphery of membrane 72 and membrane 75 are still covered by seating surfaces 65 and 69 to secure membranes 72 and 75 in the device of the present invention. Similar to embodiments described above, these embodiments secure membranes 72 and 75 into the device of the present invention, yet still provide space for membranes 72 and 75 to expand upon contact with media.

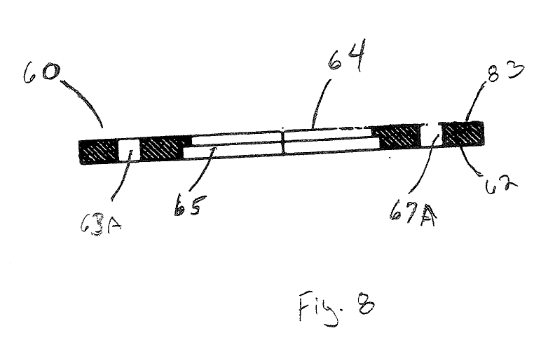

FIG. 8 shows a cross section of second member 60 along line B-B, depicted in FIG. 6. Bottom surface 62 and top surface 83 are also shown. In this embodiment, seating surface 65 is shown. As described above, the size of platform opening 64 on bottom surface 62 is larger relative to the size of platform opening 64 on top surface 83. Handling openings 63A and 67A are also shown in cross section.

In one embodiment, elevated locking surface 16 can also be positioned on second member 20. In such an embodiment, elevated locking surface 16 and locking surface opening 27 are transposed from their respective members. For example, second member 20 can optionally have elevated locking surface 16 and first member 10 can have corresponding locking surface opening 27.

The first member and the second member of the present invention can be made of any suitable material apparent to one of skill in the art. In one embodiment, the material preferably is comprised of a substantially biologically inert material such as glass, plastic or other suitable natural or synthetic polymers such as polytetrafluoroethylene.

3) The Device

As described above, an embodiment of the device of the present invention comprises a combination of a first member 10 and second member 20. In one embodiment, shown generally in FIG. 4, elevated platform surface 14 and elevated locking surface 16 of first member 10 are accommodated by openings 24 and 27 of second member 20.

However, in use (as described in more detail below), embodiments of the present invention may be combined with a membrane, such as membrane 72 shown in FIG. 7. Generally, in an embodiment wherein the device of the present invention is combined with membrane 72 (as described in more detail below), seating surface 26, in combination with protrusion 25, provides a means to secure membrane 72 between first member 10 and second member 20.

Additionally, when used with a membrane positioned between the first and second members, seating surface 26 covers an area of membrane 72 that can easily be maintained free of cells (as explained below). Therefore, seating surface 26 also protects and covers an area of membrane 72, thereby maintaining an area around the periphery of membrane 72 that is free of cells and can be readily manipulated without contaminating cells on membrane 72. Therefore, this feature provides the further advantage of reducing the risk of contamination and damage to cells positioned on membrane 72.

4) Use

In an embodiment which employs a membrane secured between first member 10 and second member 20 of the present invention, a device according to the present invention is capable of having cells spread on membrane 72. The entire device then can be placed into a vessel, such as a petri-dish, which contains appropriate cell growth media for the cells on membrane 72.

In practice, membrane 72 and optional membrane 75 are secured by the weight of second member 20 resting on membrane 72 and first member 10, thereby providing a loose fit between first member 10 with second member 20. In such an embodiment, using gravity to secure the two members together provides the advantage of facilitating the easy placement and removal of membrane 72 from between first member 10 and second member 20 because of reduced pressure on membrane 72.

In another embodiment, membrane 72 is secured by tying and/or securing clips (not shown) that can apply pressure to membrane 72 and hold first member 10 and second member 20 together. Alternatively, pressure can be applied to membrane 72 to secure membrane 72 in a device of the present invention by placing the device in a transport container or vessel that holds first member 10 and second member 20 adjacent to one another. In one embodiment, the transport container has a screw down lid, the pressure of which holds first member 10 adjacent second member 20, securing membrane 72 therebetween.

In an embodiment that uses a screw down lid, the combined height of first member 10 and second member 20, for example can be about 0.5 to 2 mm higher than the lid of the vessel, such that when the lid is screwed down, pressure on the members is increased thereby securing membrane 72 between first member 10 and second member 20. In one embodiment, first member 10 and second member 20 tightly fit together. In an embodiment with a screw down lid, a ring (not shown) on top of the device of the present invention could also be used.

However, even though a tight fit is desirable in some embodiments, when the two members fit together tightly with a membrane therebetween, for example when a method other than gravity is used to secure the members together, handling of the assembly can be more difficult in a laboratory, and assembly and disassembly of the members can also be more difficult.

In one embodiment, the spacing between the two members is chosen depending on the particular use of the device and the type of material selected to be used as membrane 72. For example, the spacing can be altered depending on the choice of the dimensions and/or physical and/or chemical characteristics of the material used for membrane 72 because different membranes expand differently. Accordingly, in some embodiments, the spacing between first member 10 and second member 20 can be increased or decreased to accommodate the expansion of different types of membranes and facilitate a desirable fit of a membrane between first member 10 and second member 20 after membrane 72 has been contacted with media. Additionally, the spacing can also be adjusted based on the choice of the material of first member 10 and second member 20.

Before or after securing membrane 72, a mixture of cells and media is slowly poured onto membrane 72 and optional membrane 75 through platform opening 24 and locking surface opening 27 of second member 20. In one embodiment, the media containing cells is poured in a specific pattern to ensure an even distribution of the cells on membrane 72. In an embodiment where membrane 75 is used, media containing cells is also poured onto membrane 75 in a specific pattern. Excess media then passes through membrane 72 and membrane 75 and out the space between first member 10 and second member 20. As the media passes through membrane 72 and membrane 75, the cells are retained on the edge of membrane 72 and membrane 75.

In one embodiment, the device of the present invention fences in or retains the cells within an intended area of membranes 72 and 75, thereby controlling the concentration of cells per square centimeter and prevent cell loss from media pouring over the device of the present invention and inadvertently carrying cells away. Because membrane 72 and membrane 75 are secured between first member 10 and seating surface 26 of second member 20, the amount of cells inadvertently carried off membrane 72 and membrane 75 or to the underside of membrane 72 or membrane 75 by the flow of the media over membrane 72 and membrane 75, is minimized. Thus, the cells are contained within the desired area until the cells have settled into or onto membrane 72 surface to allow adherence of the cells to membrane 72.

Alternatively, in an embodiment of first member 50 such as the embodiment depicted in FIG. 5, excess media can also pass through membrane 72, into openings 56A-56E and then into the vessel as it is poured onto membrane 72, as described above.

The cells in the media are captured on the edge (i.e. any intended surface of the membrane) of membrane 72 and membrane 75 and are maintained thereon. Membranes 72 and 75 are then incubated in the media at conditions suitable to permit cell growth on membranes 72 and 75.

Once appropriate cell growth is achieved, typically after a period of 1-3 days in the case of chondrocytes, the device can be removed from the vessel and first member 10 and second member 20 can be separated from each other. Once separated, membrane 75 can be examined to determine the amount of cell growth thereon.

If appropriate, membrane 72 can then be readily removed from elevated platform surface 14 because 1) the area of membrane 72 that was positioned under seating surface 26 is typically free from cell growth, and 2) any remaining area of membrane 72 that was positioned over notch 18 of elevated platform surface 14, that was not removed to form membrane 75, is also typically free from cell growth due to protrusion 25 covering a portion of membrane 72 during cell deposition and incubation.

Membrane 72 can then be transferred to another storage device for transportation or use, such as implantation into a subject, and first member 10 and second member 20 can then be sterilized and prepared for subsequent use with another membrane.

5) Specific Embodiments

In one embodiment first member 10 and second member 20 are made by cutting with machine tools. In a second embodiment, first member 10 and second member 20 are made by combining, either by gluing or melting together the separate pieces having appropriate dimensions.

a) A First Member

A polytetrafluoroethylene circular disc was provided having a diameter of 85 mm and a height of 2 mm and a rectangular elevated platform surface 14. The length L4 of the rectangular surface was 50 mm and the width W10 of the rectangular surface was 40 mm and a height H3 of 3 mm above the surface of the disc, thereby providing a first member 10 having a total height H1 of 5 mm. Elevated platform surface 14 is positioned such that two of corners of the side of the surface having a length of 41 mm were 6 mm from the edge of the circular disc. Notch 18, having dimensions of 9.5 mm by 9.5 mm, was removed from a corner of the rectangular elevated platform surface on the side of the rectangle farthest from the edge of the disc.

A square elevated locking surface 16 is also provided on first member 10. Elevated locking surface 16 has dimensions of 10 mm by 10 mm and a height H10 of 3 mm. Elevated locking surface 16 was positioned with an edge closest to the side of the elevated platform surface 14 having the notch 18. Additionally, the edge of elevated locking surface 16 was positioned about 25 mm from the center of first member 10.

b) A Second Member

A polytetrafluoroethylene circular disc is provided having a diameter of 85 mm and a height of 4.5 mm. A six-sided rectangularly shaped platform opening 24 having a complementary shape to elevated platform surface 14 of first member 10, is made in the center of the disc, thereby forming second member 20.

After platform opening 24 is formed, additional material is removed from platform opening 24 to form seating surface 26. Specifically, to a height H4 of 2.5 mm, the dimensions of platform opening 24 is expanded by 1.20 mm. The expansion of platform opening 24 forming seating surface 26 has a width WI of about 1.20 mm. Seating surface 26 is positioned circumferentially around platform opening 24 at a height H4 of 2.5 mm.

Additionally, a locking surface opening 27 is provided in second member 20 to accommodate elevated locking surface 16 of first member 10. The center of locking surface opening 27 is placed on second member 20 complementarily to the center of elevated locking surface 16 of first member 10. Accordingly, an opening having dimensions of 9 mm by 9 mm is provided, then in the same direction as was done with respect to the rectangularly shaped platform opening 24, described above, the dimensions of locking surface opening 27 were expanded by 1.20 mm to a height H4 of 2.5 mm.

Lastly, in the space remaining on second member 20 between the edge of first member 20 and the side of the platform opening 24 having the longest side, handling opening 30 is provided to facilitate handling of second member 20. Handling opening 30 is circular and had a diameter of 14 mm. Accordingly, a particular embodiment of second member 20 of the present invention was completed.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A cell loading device comprising:
a first member having at least one elevated surface;
a second member positioned adjacent said first member and having a first opening configured to accommodate said elevated surface.

2. The cell loading device of claim 1, wherein said elevated surface is configured to extend at least partially through said first opening of said second member, such that both members are removeably connected to one another.

3. The cell loading device of claim 2, wherein said first opening of said second member has a seating surface around an edge of said first opening.

4. The cell loading device of claim 2, wherein said first opening of said second member has a shape that corresponds to a shape of said elevated platform surface.

5. A method of securing a surface for cell loading comprising:
providing a first member having at least one elevated surface;
positioning a membrane on the elevated surface of the first member;
providing a second member positioned adjacent the first member and having a first opening configured to accommodate the elevated surface; and
inserting the membrane and the elevated surface of the first member into the first opening of the second member such that the first member and the second member are removably connected and the membrane is secured between the first and the second members.

6. The method of claim 5, wherein the second member has a seating surface around an edge of the first opening.

7. A method of loading cells onto a cell loading device comprising:
providing a vessel a first member having at least one elevated surface;
positioning a membrane on the platform surface of the first member;
providing a second member positioned adjacent the first member and having a first opening configured to accommodate the elevated surface;
inserting the membrane and the elevated surface of the first member into the first opening of the second member such that the first member and the second member are removably connected and the membrane is secured between the first and the second members; and
loading a suspension of cells on the membrane through the opening on the top surface of the second member.

8. The method of claim 7, wherein the second member has a seating surface around an edge of the first opening.

9. The device of claim 1, wherein said platform surface has at least one opening.

10. The device of claim 1, wherein at least one of said first and second members further comprises an elevated locking surface.

11. The device of claim 10, wherein the other of said first and second members further comprises an opening configured to receive said elevated locking surface.

12. The device of claim 1, wherein one of said first and second members has at least one handling opening.
